(19) United States
(12) Patent Application Publication
(10) Pub. No.: US 2007/0027240 A1
(43) Pub. Date: Feb. 1, 2007

(54) Diethyleneglycol ester based plasticizer composition and polyvinyl chloride resin using the same

(76) Inventors: Kye-Seok Lee, Daejeon-city (KR); Kyu-Il Lee, Daejeon-city (KR); Hyun-Kyu Kim, Daejeon-city (KR); Chil-Eui Hong, Daejeon-city (KR); Dong-Hwa Gong, Daejeon-city (KR); Hyun-Ju Kim, Daejeon-city (KR)

Correspondence Address: CANTOR COLBURN, LLP, 55 GRIFFIN ROAD SOUTH, BLOOMFIELD, CT, 06002, US

(21) Appl. No.: 10/570,960

(22) PCT Filed: Sep. 9, 2004
(86) PCT No.: PCT/KR04/02307

§ 371 Date: Mar. 8, 2006

(30) Foreign Application Priority Data
Sep. 9, 2003 (KR) 10 2003- 0063160

(51) Int. Cl. C08F 14/06; C07C 69/76; C08K 5/17

(52) U.S. Cl. 524/239; 526/344; 560/81

(57) ABSTRACT

The present invention relates to a diethylene glycol ester based plasticizer composition comprising at least two diethylene glycol ester compounds. The diethylene glycol ester based plasticizer composition of the present invention offers superior tensile strength, elongation, transfer resistance, hardness, transparency, adhesivity, and compatibility with resin, when used for a plasticizer of a polyvinyl chloride resin, without releasing environmental hormones. Thus, it can be used for a polyvinyl chloride resin of a warp film.

### TECHNICAL FIELD

The present invention relates to a diethyleneglycol ester based plasticizer composition, and more particularly to a diethyleneglycol ester based plasticizer composition which can be used as plasticizer of a polyvinyl chloride (PVC) resin.

### BACKGROUND ART

The plasticizer added to a polyvinyl chloride resin or other polymer resins is an essential additive offering the polymer resin physical properties and capabilities such as processability, flexibility, electrical insulation, adhesivity, etc. Conventionally, phthalates, adipates, etc. have been typically used as plasticizer. The most widely used plasticizers are di-2-ethylhexylphthalate (DEHP) and di-2-ethylhexylalipate (DEHA). These plasticizers are used as standards for evaluating other plasticizers.

### DISCLOSURE OF INVENTION

Technical Problem

However, the Environmental Protection Agency (EPA) of the U.S. and the National Institute of Health Sciences of Japan have classified phthalates and adipates as environmental hormone materials.

Thus, development of a plasticizer comprising neither phthalate nor adipate is required. U.S. Pat. No. 5,746,783 discloses a diesel fuel additive comprising a di-ethyleneglycol ester based compound, which comprises neither phthalate structure nor adipate structure and reduces nitrogen oxide exhausted from diesel engine.

A polyvinyl chloride resin is prepared from vinyl chloride monomers and other monomers copolymerizable with the vinyl chloride monomers. By adding such additives as a plasticizer, a stabilizer, a filler, a pigment, etc., the resin can be imbued with a variety of processing properties.

The polyvinyl chloride resin is used for numerous applications including pipes, cables, artificial leather, wallpaper, gloves, toys, wrap films, etc.

Particularly, the polyvinyl chloride resin used for a wrap film for packing foods requires good tensile strength, hardness, elongation capable of wrapping well packing manufactures, transparency capable of seeing well with the naked eye foods, adhesivity after foods packing, transfer resistance, and compatibility with other resins.

Technical Solution

It is an aspect of the present invention to provide a diethylene glycol ester compound for a plasticizer not releasing environmental hormones.

It is another aspect of the present invention to provide a diethylene glycol ester based plasticizer composition useful for a plasticizer of a polyvinyl chloride resin comprising a diethylene glycol ester compound.

It is still another aspect of the present invention to provide a polyvinyl chloride resin for a wrap film having superior tensile strength, elongation, hardness, transfer resistance, transparency, and adhesivity using the diethylene glycol ester based plasticizer composition.

In order to attain the aspects, the present invention provides a diethylene glycol ester compound for a plasticizer represented by Formula 1-1 below:
R₁OCO—(CH₂)₂—O—(CH₂)₂—OCOR₂ (1-1)

wherein R₁ is a phenyl group and R₂ is an alkyl group having 3 to 12 carbon atoms.

The present invention also provides a diethylene glycol ester based plasticizer composition comprising the diethylene glycol ester compound represented by Formula 1-1.

The composition further comprises a diethylene glycol ester compound represented by Formula 1-2 below; and a diethylene glycol ester compound represented by Formula 1-3 below.
R₃ OCO—(CH₂)₂—O—(CH₂)₂—OCOR₄ (1-2)

(wherein each of R₃ and R₄ is an alkyl group having 3 to 12 carbon atoms.)
R₅OCO—(CH₂)₂—O—(CH₂)₂—OCOR₆ (1-3)

(wherein each of R₅ and R₆ is a phenyl group.)

More preferably, the plasticizer composition of the present invention comprises:

(a) 10 to 80 wt % of the diethylene glycol ester compound represented by Formula 1-1;

(b) 5 to 80 wt % of the diethylene glycol ester compound represented by Formula 1-2; and

(c) 5 to 60 wt % of the diethylene glycol ester compound represented by Formula 1-3.

The present invention also provides a method of preparing a diethylene glycol ester based plasticizer composition by esterifiying 10 to 40 wt % of diethylene glycol, 20 to 70 wt % of 2-ethylhexanoic acid, 10 to 60 wt % of benzoic acid 1 to 10 wt % of xylene,

The present invention also provides a polyvinyl chloride resin prepared using the diethylene glycol ester based plasticizer composition.

Advantageous Effects

As described above, the diethylene glycol ester based plasticizer composition of the present invention improves tensile strength, elongation, transfer resistance, hardness, transparency, adhesivity, and/or compatibility of a polyvinyl chloride resin without generating environmental hormones, when employed as the plasticizer of the resin.

### BRIEF DESCRIPTION OF THE DRAWINGS

FIG. 1 shows a gas chromatography mass spectrometer (GC-MS) for diethylene glycol ester based plasticizer composition of Example 1 according to the present invention.

FIG. 2 shows a gas chromatography mass spectrometer (GC-MS) for diethylene glycol ester based plasticizer composition of Example 2 according to the present invention.

FIG. 3 shows a gas chromatography mass spectrometer (GC-MS) for diethylene glycol ester based plasticizer composition of Example 3 according to the present invention.

### BEST MODE FOR CARRYING OUT THE INVENTION

Hereinafter, the present invention is described in more detail.

The present invention is characterized by a plasticizer composition not comprising phthalate or adipate, which are known as environmental hormone materials and having superior tensile strength, elongation, hardness, transfer resistance, transparency and adhesivity, thereby being useful for a polyvinyl chloride resin for a wrap film for packing foods.

Preferably, the diethylene glycol ester based plasticizer composition of the present invention comprises at least two selected from the group consisting of diethylene glycol ester compounds represented by Formula 1 below.
R₇OCO—(CH₂)₂—O—(CH₂)₂—OCOR₈ (1)

wherein each of R₇ and R₈ is a phenyl group or an alkyl group having 3 to 12 carbon atoms.

A wrap film used for packing foods should have superior tensile strength, hardness, transfer resistance, transparency, elongation, and adhesivity. A plasticizer is added to the resin formed the foundation of the wrap film to offer such physical properties. The present invention uses the compound represented by Formula 1 as plasticizer.

However, all of above physical properties cannot be satisfied only with the compound represented by Formula 1. To be specific, the compound of Formula 1 is 2-ethylhexanoic acid 2-2-(2-ethylhexanoyloxy)-ethoxy-ethyl ester synthesized from di-ethylglycol and 2-ethylhexanoic acid, or benzoic acid 2-2-(benzoyloxy)-ethoxy-ethyl ester synthesized from diethylglycol and benzoic acid. However, the compounds of Formula 1 are used more as lubricant than as plasticizer, because they are not sufficiently compatible with polyvinyl chloride resin. And, 2-ethylhexanoic acid 3-(2-ethylhexanoyloxy)-2,2-dimethylpropyl ester synthesized from neopentylglycol and 2-ethylhexanoic acid as other compound of Formula 1 is insufficient for a wrap film because of high hardness and low transparency and elongation. Also, although 2-ethylhexanoic acid [2-2-(2-ethylhexanoyloxy)-ethoxyethoxy]-ethyl ester synthesized from triethylene glycol and 2 ethylhexanoic acid has superior processability, it is disadvantageous in such physical properties as elongation, adhesivity, transparency, etc.

However, if two or more diethylene glycol ester compounds selected from the compounds represented by Formula 1 are used in different proportions, plasticizers with different physical properties are obtained.

The plasticizer composition of the present invention offers superior tensile strength, elongation, hardness, transfer resistance, transparency, and adhesivity and has superior processability including compatibility with polyvinyl chloride resin by using two or more diethylene glycol ester compounds selected from the compounds represented by Formula 1.

According to the method of preparing the diethylene glycol ester based plasticizer composition of the present invention, at least two compounds selected from the compounds represented by Formula 1, preferably three diethylene glycol ester compounds, are produced.

Of such compounds represented by Formula 1, the present invention is characterized by a diethylene glycol ester compound having the structure of Formula 1-1 below:
R₁OCO—(CH₂)₂—O—(CH₂)₂—OCOR₂ (1-1)

wherein R₁ is a phenyl group and R₂ is an alkyl group having 3 to 12 carbon atoms.

Because the compound represented by Formula 1-1 is the most preferable in terms of stability, a plasticizer composition comprising it has superior physical properties as plasticizer of a polyvinyl chloride resin.

Accordingly, the present invention is characterized by a plasticizer composition of a polyvinyl chloride resin comprising the diethylene glycol ester compound represented by Formula 1-1.

Preferably, the composition of the present invention comprises the diethylene glycol ester compounds represented by Formulas 1-2 and 1-3 below as the compound represented by Formula 1.
R₃OCO—(CH₂)₂—O—(CH₂)₂—OCOR₄ (1-2)

wherein each of R₅ and R₆ is an alkyl group having 3 to 12 carbon atoms.
R₅OCO—(CH₂)₂—O—(CH₂)₂—OCOR₆ (1-3)

wherein each of R₅ and R₆ is a phenyl group.

Preferably, the plasticizer composition of the present invention comprises:

(a) 10 to 80 wt % of the diethylene glycol ester compound represented by Formula 1-1;

(b) 5 to 80 wt % of the diethylene glycol ester compound represented by Formula 1-2; and

(c) 5 to 60 wt % of the diethylene glycol ester compound represented by Formula 1-3.

Preferably, (a) the compound represented by Formula 1-1 is benzoic acid 2-{2-(2-ethylhexanoyloxy)-ethoxy}-ethyl ester.

And, preferably, (b) the compound represented by Formula 1-2 is 2-ethylhexanoic acid 2-{2-(2-ethylhexanoyloxy)-ethoxy}-ethyl ester.

And, preferably, (c) the compound represented by Formula 1-3 is benzoic acid 2-{2-(benzoyloxy)-ethoxy}-ethyl ester.

As a preferred embodiment of the present invention, the diethylene glycol ester based plasticizer composition may comprise 10 to 80 wt % of benzoic acid 2-{2-(2-ethylhexanoyloxy)-ethoxy}-ethyl ester; 5 to 80 wt % of 2-ethylhexanoic acid 2-{2-(2-ethylhexanoyloxy)-ethoxy}-ethyl ester; and 5 to 60 wt % of benzoic acid 2-{2-(benzoyloxy)-ethoxy}-ethyl ester.

More preferably, the diethylene glycol ester based plasticizer composition of the present invention comprises 30 to 60 wt % of benzoic acid 2-{2-(2-ethylhexanoyloxy)-ethoxy}-ethyl ester; 10 to 60 wt % of 2-ethylhexanoic acid 2-{2-(2-ethylhexanoyloxy)-ethoxy}-ethyl ester; and 5 to 50 wt % of benzoic acid 2-{2-(benzoyloxy)-ethoxy}-ethyl ester.

Most preferably, the composition of the present invention comprises the compounds (a), (b), and (c) at a proportion of 40:40:20 based on weight.

Hereinafter, the preparing method of the diethylene glycol ester based plasticizer composition of the present invention is described in more detail.

(a) The benzoic acid 2-{2-(2-ethylhexanoyloxy)-ethoxy}-ethyl ester may be synthesized from diethylene glycol, 2-ethylhexanoic acid and benzoic acid by esterification.

(b) The 2-ethylhexanoic acid 2-{2-(2-ethylhexanoyloxy)-ethoxy}-ethyl ester may be synthesized from diethylene glycol and 2-ethylhexanoic acid by esterification.

And, (c) the benzoic acid 2-{2-(benzoyloxy)-ethoxy}-ethyl ester may be synthesized from diethylene glycol and benzoic acid by esterification.

To sum up, the diethylene glycol ester based plasticizer composition of the present invention may be prepared from diethylene glycol, benzoic acid, and 2-ethylhexanoic acid. In addition, other additives may be used.

In a preferred embodiment of the present invention, the diethylene glycol ester plasticizer composition is prepared as follows.

10 to 40 wt % of diethylene glycol, 20 to 70 wt % of 2-ethylhexanoic acid, 10 to 60 wt % of benzoic acid, 1 to 10 wt % of xylene as entrainer, and 0.05 to 1 wt % of tetra-isopropyl titanate as catalyst, are put in a flask equipped with a stirrer and a condenser. The temperature of the flask is raised to 220° C. and esterification is performed for 4 to 10 hours. After the esterification is over, the unreacted acid is removed with a vacuum pump and the remaining acid is neutralized with 5 to 15 wt % of sodium hydroxide.

After washing with water, dehydrating, and filtering, a diethylene glycol ester plasticizer composition comprising 30 to 60 wt % of benzoic acid 2-{2-(2-ethylhexanoyloxy)-ethoxy} ethyl ester, 10 to 60 wt % of 2-ethylhexanoic acid 2-{2-(2-ethylhexanoyloxy)-ethoxyl}-ethyl ester, and 5 to 40 wt % of benzoic acid 2-{2-(benzoyloxy)-ethoxy} ethyl ester is obtained.

The resultant diethylene glycol ester plasticizer composition can be used as plasticizer of a polyvinyl chloride resin to improve tensile strength, elongation, hardness, transfer resistance, transparency, adhesivity, and/or compatibility of the resin.

The diethylene glycol ester based plasticizer composition of the present invention may also be used for other resins to improve tensile strength, elongation, transfer resistance, hardness, transparency, and/or adhesivity, and it is particularly suitable for a polyvinyl chloride resin of a wrap film used for packing foods. It may also be used for a polyethylene based foaming sheet.

Thus, the present invention provides a polyvinyl chloride resin using the above-described diethylene glycol ester based plasticizer composition.

The method of preparing the polyvinyl chloride resin is not particularly limited. It can be prepared by the conventional method of copolymerizing polyvinyl chloride with monomers copolymerizable with polyvinyl chloride using the plasticizer composition.

Hereinafter, the present invention is described in more detail through examples.

However, the following examples are only for the understanding of the present invention and they do not limit the present invention.

### EXAMPLES

### Example 1

(1) Preparation of Diethylene Glycol Ester Plasticizer Composition

3 moles of diethylene glycol, 5.4 moles of 2-ethylhexanoic acid, 2.4 moles of benzoic acid, 60 g of xylene as entrainer, and 2 g of tetraisopropyl titanate as catalyst, were put in a 2-L round flask equipped with a stirrer and a condenser. The temperature was raised to 220° C. and esterification was performed for 10 hours.

Unreacted acid was removed with a vacuum pump at 220° C. and 2 mmHg, and remaining acid was neutralized with 10 wt % of sodium hydroxide. After washing with water and dehydrating, an absorbent was added. The resultant solution was filtered to obtain a diethylene glycol ester composition.

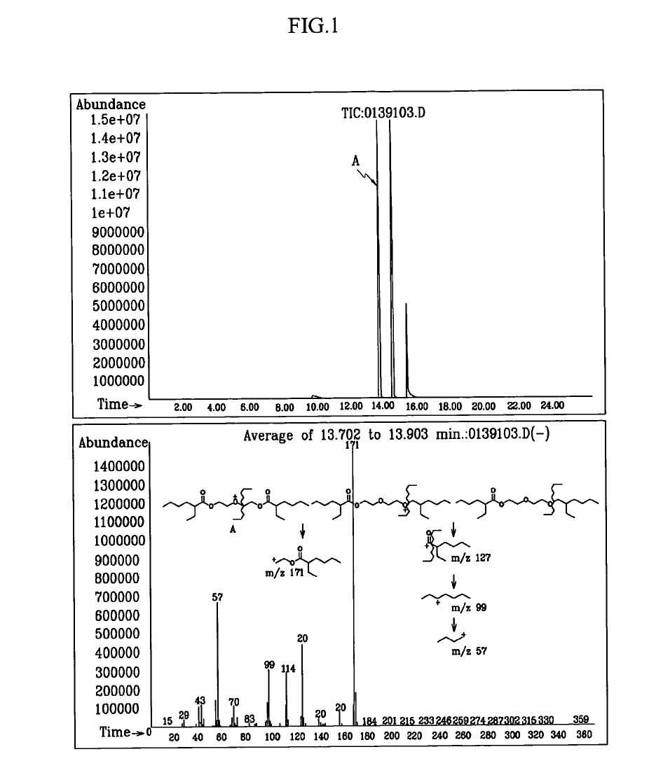

The diethylene glycol ester composition comprised 45 wt % of benzoic acid 2-{2-(2-ethylhexanoyloxy)-ethoxy} ethyl ester, 42 wt % of 2-ethylhexanoic acid 2-{2-(2-ethylhexanoyloxy)-ethoxy}-ethyl ester, 12 wt % of benzoic acid 2-{2-(benzoyloxy)-ethoxy} ethyl ester, and 1 wt % of other unreacted materials. The composition and the content were confirmed with a gas chromatography mass spectrometer (GC-MS). The GC-MS result is shown in FIG. 1.

(2) Preparation of Polyvinyl Chloride Resin Using Diethylene Glycol Ester Plasticizer Composition

36 parts by weight of the diethylene glycol ester composition prepared in (1), 1.3 part by weight of a calcium-zinc stabilizer (KCZ-08, Kolon) and 12 parts by weight of epoxylated soybean oil (ShinDongBang) per 100 parts by weight of a polyvinyl chloride resin (LS100S, LG Chem) were nixed and prepared sheet a thickness of 5-mm using a roll mill at 165° C. for 3 minutes. The sheet was pressed to a thickness of 1-mm. The pressing conditions included preheating at 185° C. for 3 minutes, heating for 3 minutes, and cooling for 3 minutes. The resultant sheet having a thickness of 1-mm was prepared into several type C dumbbell shaped samples.

(3) Physical Property Test of Polyvinyl Chloride Resin

Tensile strength, elongation, hardness, transfer resistance, transparency, and adhesivity were evaluated for the samples prepared in (2). The result is given in Table 1 below.

* Tensile strength: Tested according to ASTM D638 using a U.T.M. The sample was pulled at a cross head speed of 500 mm/min. The site where the sample was cut was checked and the tensile strength was calculated by the following equation.
Tensile strength (kgf/mm²)=load (kgf)/(thickness (mm)×width (mm)) Equation [1]

* Elongation: Tested according to ASTM D638 using a U.T.M. The sample was pulled at a cross head speed of 500 mm/min. The site where the sample was cut was checked and the elongation was calculated by the following equation.
Elongation (%)=(extension/initial strength)×100 Equation [2]

* Hardness: Hardness was tested at 5 sites for each sample to obtain the mean value.

* Transfer resistance: The initial weight (Wᵢ) of the sample was weighed to the fourth decimal point. The sheet sample (3 cm z, 3 cm) was inserted between polystyrene (PS) plates and put in an oven of 80° C. After letting for 48 hours under a load of 1 kg, the sample was taken out of the oven and kept in a constant temperature bath for at least 4 hours. The weight (Wₒ) of the sample was weighed and the degree of transfer was calculated by the following equation.
Degree of transfer (%)=(Wᵢ−Wₒ)/Wᵢ×100 Equation [3]

* Transparency: If the transparency was better than that of the di-2-ethylhexyladipate (DEHA), a standard plasticizer, when observed with naked eyes, the transparency was evaluated as superior. If the transparency was comparable to that of DEHA, it was evaluated as comparable, and if it was inferior to that of DEHA, it was evaluated as inferior.

* Adhesivity: If the adhesivity was better than that of DEHA, when touched with a hands the adhesivity was evaluated as superior. If the adhesivity was comparable to that of DEHA, it was evaluated as comparable, and if it was inferior to that of DEHA, it was evaluated as inferior.

### Example 2

A diethylene glycol ester composition was prepared in the same manner of Example 1, except for changing the composition and content as shown in Table 1. A polyvinyl chloride resin was prepared using the composition as plasticizer. Tensile strength, elongation, hardness, transfer resistance, transparency, and adhesivity were evaluated as in Example 1. The result is given in Table 1.

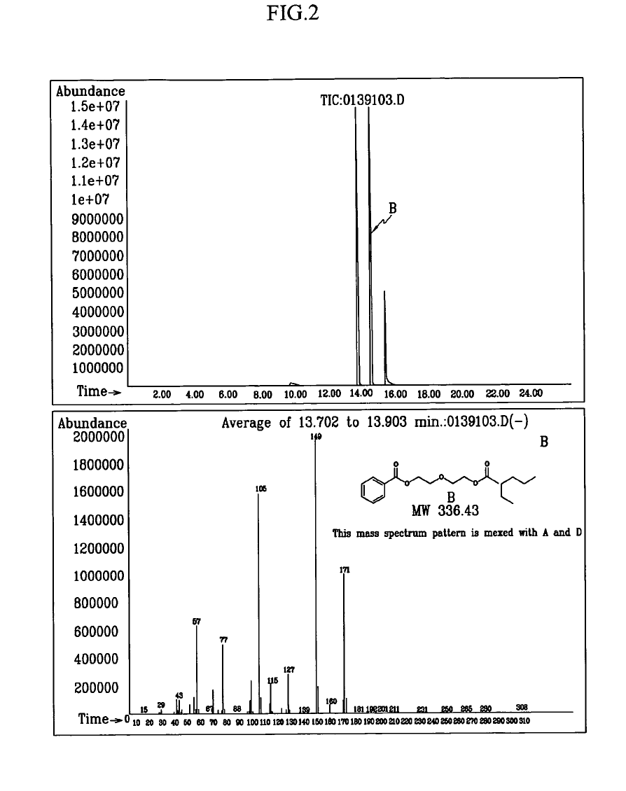

The diethylene glycol ester composition comprised 50 wt % of benzoic acid 2-{2-(2-ethylhexanoyloxy)-ethoxy}-ethyl ester, 24 wt % of 2-ethylhexanoic acid 2-{2-(2-ethylhexanoyloxy)-ethoxy}-ethyl ester, 24 wt % of benzoic acid 2-{2-(benzoyloxy)-ethoxy}-ethyl ester, and 2 wt % of other unreacted materials. The composition and the content were confirmed with a gas chromatography mass spectrometer (GC-MS). The GC-MS result is shown in FIG. 2.

### Example 3

A diethylene glycol ester composition was prepared in the same manner of Example 1, except for changing the composition and content as shown in Table 1. A polyvinyl chloride resin was prepared using the composition as plasticizer. Tensile strength, elongation, hardness, transfer resistance, transparency, and adhesivity were evaluated as in Example 1. The result is given in Table 1.

The diethylene glycol ester composition comprised 42 wt % of benzoic acid 2-{2-(2-ethylhexanoyloxy)-ethoxy}-ethyl ester, 12 wt % of 2-ethylhexanoic acid 2-{2-(2-ethylhexanoyloxy)-ethoxy}-ethyl ester, 44 wt % of benzoic acid 2-{2-(benzoyloxy)-ethoxy} ethyl ester, and 2 wt % of other unreacted materials. The composition and the content were confirmed with a gas chromatography mass spectrometer (GC-MS).

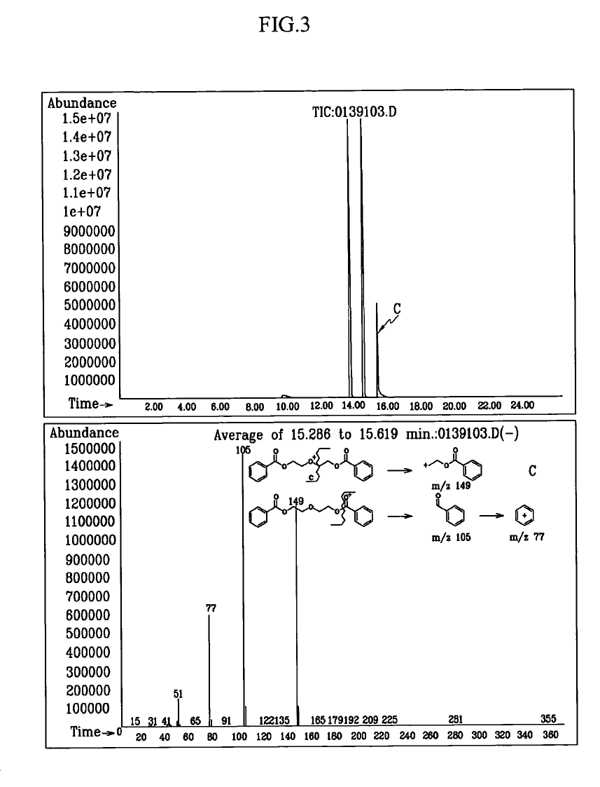

The GC-MS result is shown in FIG. 3.

### Comparative Example 1

A polyvinyl chloride resin was prepared in the same manner of Example 1, except for using di-2-ethylhexyladipate (DOA, LG Chem) instead of diethylene glycol ester of the present invention as plasticizer. Tensile strength, elongation, hardness, transfer resistance, transparency, and adhesivity were evaluated. The result is given in Table 1.

### Comparative Example 2

A polyvinyl chloride resin was prepared in the same manner of Example 1, except for using an ester based plasticizer prepared from neopentylglycol and 2-ethylhexanoic acid instead of diethylene glycol ester of the present invention as plasticizer. Tensile strength, elongation, hardness, transfer resistance, transparency, and adhesivity were evaluated. The result is given in Table 1.

### Comparative Example 3

A polyvinyl chloride resin was prepared in the same manner of Example 1, except for using an ester based plasticizer prepared from triethylene glycol and 2-ethylhexanoic acid instead of diethylene glycol ester of the present invention as plasticizer. Tensile strength, elongation, hardness, transfer resistance, transparency, and adhesivity were evaluated. The result is given in Table 1.

### Comparative Example 4

A polyvinyl chloride resin was prepared in the same manner of Example 1, except for using diisonoyladipate (DINA, LG Chem) instead of diethylene glycol ester of the present invention as plasticizer. Tensile strength, elongation, hardness, transfer resistance, transparency, and adhesivity were evaluated. The result is given in Table 1.

TABLE 1

| | | Example | | | Comparative Example | | | |
|---|---|---|---|---|---|---|---|---|
| | | 1 | 2 | 3 | 1 | 2 | 3 | 4 |
| Composition | Diethylene | 3(666) | 3(666) | 3(666) | — | — | — | — |
| | glycolmol (g) | | | | | | | |
| | 2-Ethylhexanoic | 5.4(1.296) | 3.3(792) | 2.1(504 | — | 3.1(744) | 2.2(528) | — |
| | acidmol (g) | | | | | | | |
| | Benzoic | 2.4(446.4) | 3.3(613.8) | 4.5(837) | — | — | — | — |
| | acidmol (g) | | | | | | | |
| | Neopentylglycolmol (g) | — | — | — | — | 1.2(124.8) | — | — |
| | Triethyleneglycolmol (g) | — | — | — | — | — | 1(150) | — |
| | Xylene (g) | 60 | 60 | 60 | — | 60 | 60 | — |
| | Tetraisopropyl | 2 | 2 | 2 | — | 1.5 | 2 | — |
| | titanate (g) | | | | | | | |

| | | | | | | | |
|---|---|---|---|---|---|---|---|
| Tensile strength | 1.91 | 1.81 | 1.85 | 1.81 | 1.87 | 1.84 | 1.73 |
| (kgf/mm²) | — | — | — | — | — | — | — |
| Elongation (%) | 390 | 382 | 380 | 380 | 385 | 348 | 371 |
| Transfer resistance (%) | 1.95 | 2.21 | 3.22 | 0.2 | 0.2 | 0.3 | 0.1 |
| Hardness (R-scale) | 75 | 75 | 75 | 78 | 76 | 78 | 76 |
| Transparency | Superior | Superior | Superior | Standard | Comparable | Inferior | Comparable |
| Adhesivity | Superior | Comparable | Comparable | Standard | Inferior | Inferior | Inferior |

As seen in Table 1, the polyester resin using the diethylene glycol ester composition of the present invention as plasticizer (Example 1) showed better tensile strength, elongation, transparency, and adhesivity and comparable hardness, compared with those prepared using the DOA (Comparative Example 1), the conventional plasticizer most widely used in a wrap film for packing foods, the DINA (Comparative Example 4), which has the structure similar to that of DEHA, the standard plasticizer, the single-component plasticizer prepared from neopentylglycol and 2-ethylhexanoic acid (Comparative Example 2) and the single-component plasticizer prepared from triethylene glycol and 2-ethylhexanoic acid (Comparative Example 3). Thus, the plasticizer of Example 1 is suitable for a plasticizer of a wrap film. To process a wrap film, the transfer resistance should not be too high or too low. The plasticizer of Example 1 offers a suitable transfer resistance for a wrap film.

And, the polyester resins using the diethylene glycol ester compositions of Examples 2 and 3 as plasticizer have comparable, although not significantly superior, physical properties, compared with those of Comparative Examples. These physical properties are adequate for a wrap film. More importantly, they do not comprise phthalate, adipate, or trimelitate compounds, which are known to generate environmental hormones.

As described above, the diethylene glycol ester based plasticizer composition of the present invention improves tensile strength, elongation, transfer resistance, hardness, transparency, adhesivity, and/or compatibility of a polyvinyl chloride resin without generating environmental hormones, when employed as the plasticizer of the resin.

While the present invention has been described in detail with reference to the preferred embodiments, those skilled in the art will appreciate that various modifications and substitutions can be male thereto without departing from the spirit and scope of the present invention as set forth in the appended claims.

1. A diethylene glycol ester compound for a plasticizer represented by Formula 1-1 below:
R₁OCO—(CH₂)₂—O—(CH₂)₂—OCOR₂ (1-1)
wherein R₁ is a phenyl group and R₂ is an alkyl group having 3 to 12 carbon atoms.

2. The diethylene glycol ester compound of claim 1, wherein the diethylene glycol ester comprises benzoic acid 2-{2-(2-ethylhexanoyloxy)-ethoxy}-ethyl ester.

3. The diethylene glycol ester compound of claim 2, wherein the benzoic acid 2-{2-(2-ethylhexanoyloxy)-ethoxy}-ethyl ester is prepared from esterification of diethylene glycol, 2-ethylhexanoic acid, and benzoic acid.

4. A diethylene glycol ester based plasticizer composition comprising a diethylene glycol ester compound represented by Formula 1-1:
R₁OCO—(CH₂)₂—O—(CH₂)₂—OCOR₂ (1-1)
wherein R₁ is a phenyl group and R₂ is an alkyl group having 3 to 12 carbon atoms.

5. The diethylene glycol ester based plasticizer composition of claim 4, which further comprises:
a diethylene glycol ester compound represented by Formula 1-2 below: and
a diethylene glycol ester compound represented by Formula 1-3,
R₃OCO—(CH₂)₂—O—(CH₂)₂—OCOR₄ (1-2)
wherein each of R₃ and R4 is an alkyl group having 3 to 12 carbon atoms; and
R₅OCO—(CH₂)₂—O—(CH₂)₂—OCOR₆ (1-3)
wherein each of R₅ and R6 is a phenyl group.

6. The diethylene glycol ester based plasticizer composition of claim 4, further comprising:
(a) 10 to 80 wt % of the diethylene glycol ester compound represented by Formula 1-1;
(b) 5 to 80 wt % of the diethylene glycol ester compound represented by Formula 1-2; and
(c) 5 to 60 wt % of the diethylene glycol ester compound represented by Formula 1-3.

7. The diethylene glycol ester based plasticizer composition of claim 6, wherein:
(a) the compound represented by Formula 1-1 is benzoic acid 2-{2-(2-ethylhexanoyloxy)-ethoxy}-ethyl ester,
(b) the compound represented by Formula 1-2 is 2-ethylhexanoic acid 2-{2-(2-ethylhexanoyloxy) -ethoxy}-ethyl ester; and
(c) the compound represented by Formula 1-3 is benzoic acid 2-{2-(benzoyloxy)-ethoxy}-ethyl ester.

8. A method of preparing a diethylene glycol ester based plasticizer composition comprising esterifying 10 to 40 wt % of diethylene glycol, 20 to 70 wt % of 2-ethylhexanoic acid, 10 to 60 wt % of benzoic acid, 1 to 10 wt % of xylene, and 0.01 to 1 wt % of tetraisopropyl titanate at 220° C. for 4 to 10 hours.

9. The preparing method of claim 8, wherein the composition comprises:
(a) 10 to 80 wt % of the diethylene glycol ester compound represented by Formula 1-1,
R₁OCO—(CH₂)₂—O—(CH₂)₂—OCOR₂ (1-1);
(b) 5 to 80 wt % of the diethylene glycol ester compound represented by Formula 1-2,
R₃OCO—(CH₂)₂—O—(CH₂)₂—OCOR₄ (1-2); and
(c) 5 to 60 wt % of the diethylene glycol ester compound represented by Formula 1-3,
R₅OCO—(CH₂)₂—O—(CH₂)₂—OCOR₆ (1-3).

10. A polyvinyl chloride resin prepared using any one diethylene glycol ester based plasticizer composition of claim.
